(19) United States
(12) Patent Application Publication
(10) Pub. No.: US 2026/0140045 A1
(43) Pub. Date: May 21, 2026

(54) SYSTEM AND METHOD TO RETARD H2S REACTION WITH A TARGET SENSING ELEMENT

(71) Applicant: Halliburton Energy Services, Inc., Houston, TX (US)

(72) Inventors: Christopher Michael Jones, Houston, TX (US); James Martin Price, Houston, TX (US)

(73) Assignee: Halliburton Energy Services, Inc., Houston, TX (US)

(21) Appl. No.: 18/949,710

(22) Filed: Nov. 15, 2024

(51) Int. Cl. G01N 21/31; E21B 49/10; G01N 33/28

(52) U.S. Cl. CPC G01N 21/31; E21B 49/10; G01N 33/2823

(57) ABSTRACT

Disclosed herein are methods and systems for fluid analysis and, more particularly, for identifying and measuring the concentration of H₂S in real-time downhole during the fluid analysis. The methods may include conveying a fluid sampling tool into a wellbore, drawing a formation fluid from a sampling zone, and passing the fluid sample over a H₂S gas diffusion barrier that controls H₂S diffusion from the fluid sample to a thin film deposited on a measurement region, wherein the measurement region is part of the at least one passageway, and analyzing the fluid sample in the fluid sampling tool for a target analyte.

### BACKGROUND

During oil and gas exploration, many types of information may be collected and analyzed. This information may be used to determine the quantity and quality of hydrocarbons in a reservoir and to develop or modify strategies for hydrocarbon production. For instance, the information may be used for reservoir evaluation, flow assurance, reservoir stimulation, facility enhancement, production enhancement strategies, and reserve estimation. One technique for collecting relevant information involves obtaining and analyzing fluid samples from a reservoir of interest. There are a variety of different tools that may be used to obtain the fluid sample. The fluid sample may then be analyzed to determine fluid properties.

It is often desired to collect a representative sample of formation or reservoir fluids (e.g., hydrocarbons) to further evaluate drilling operations and production potential, or to detect the presence of certain gases or other materials in the formation that may affect well performance. For example, hydrogen sulfide (H₂S), a poisonous, corrosive, and flammable gas, can occur in formation fluids, and its presence in the wellbore in significant concentrations may result in damage to wellbore components or dangerous conditions for well operators at the surface. However, H₂S concentration in formation fluids are often underestimated with current measurement techniques due to losses via absorption/adsorption or H₂S scavenging on tool surfaces, sampling bottle, and/or during sample transfers, for example. Additionally, downhole measurements of H₂S are not currently performed in real-time, which may prevent personnel from identifying potential hazards from H₂S before an accident may occur.

### BRIEF DESCRIPTION OF THE DRAWINGS

These drawings illustrate certain aspects of some of the embodiments of the present disclosure, and should not be used to limit or define the disclosure:

FIG. 1 is a schematic diagram of an example of a fluid sampling tool on a wireline;

FIG. 2 is a schematic diagram of an example of the fluid sampling tool on a drill string;

FIG. 3 is a schematic of an information handling system;

FIG. 4 is a schematic of a chipset that may be utilized by the information handling system;

FIG. 5 is a schematic of an arrangement of resources on a computer network;

FIG. 6 is a schematic of a fluid sampling tool;

FIG. 7 depicts one design of the hardware configuration of the dynamic subsurface optical measurement tool according to embodiments of the present disclosure;

FIG. 8A depicts a design of a H₂S gas diffusion barrier deposited over a thin metal film deposited on a substrate in a measurement region according to embodiments of the present disclosure.

FIG. 8B depicts another design of a H₂S gas diffusion barrier deposited over a thin metal film deposited on a substrate in a measurement region according to embodiments of the present disclosure.

FIG. 8C depicts another design of a H₂S gas diffusion barrier deposited over a thin metal film deposited on a substrate in a measurement region according to embodiments of the present disclosure.

FIG. 9 depicts a bridge design in the hardware configuration of the dynamic subsurface optical measurement tool according to embodiments of the present disclosure.

FIG. 10 illustrates an enlargement of the bridge design according to embodiments of the present disclosure.

FIG. 11 illustrates a workflow to determine the presence of hydrogen sulfide (H₂S) according to embodiments of the present disclosure.

### DETAILED DESCRIPTION

Disclosed herein are methods and systems for fluid analysis and, more particularly, are disclosed methods and systems for identifying and measuring the concentration of H₂S in real-time downhole during the fluid analysis. Methodologies in accordance with the present disclosure use a downhole spectroscopy device with a fluid sampling device to determine the presence and concentration of H₂S. Optical and/or electrical measurement systems and methods may be utilized to quantify H₂S downhole. Specifically, the use of a H₂S gas diffusion barrier may control the diffusion of H₂S from the fluid in the flow line towards materials that react with H₂S may be used in a fluid sampling tool to measure the concentration of H₂S in real time during fluid analysis and sampling operations. The materials that react with H₂S include thin metal oxide such as tin oxide alloyed doped with silver, chromium oxide alloyed doped with silver, indium tin oxide alloyed doped with silver, and any combination thereof. The thin metal oxide may be from about 3 nm to about 100 nm thick, from about 5 nm to about 75 nm thick, from about 10 nm to about 50 nm thick, or from about 15 nm to about 25 nm thick, for example. The H₂S gas diffusion barrier that slows down the rate of diffusion of H₂S and its reaction with the H₂S sensitive material includes beryllium oxide, thin dielectric, semiconductor thin film with a lattice constant smaller than H₂S, and any combination thereof. The H₂S gas diffusion barrier may be from about 1 nm to about 1000 nm thick, from about 5 nm to about 500 nm thick, from about 10 nm to about 250 nm thick, from about 20 nm to about 200 nm thick, from about 25 nm to about 100 nm thick, or from about 50 nm to about 75 nm thick, for example.

There may be one or more zones of H₂S sensitive material without any H₂S gas diffusion barrier and one or more zones of H₂S sensitive material covered by the H₂S gas diffusion barrier to slow down H₂S diffusion and its reaction with the H₂S sensitive material. Further, the H₂S gas diffusion barrier may be applied in different concentration and/or thickness over the H₂S sensitive material for the H₂S sensitive material to be sensitive to different concentration of H₂S. In embodiments, the H₂S sensitive material may cover the optical path of the downhole spectroscopy device only partially. Then, the H₂S sensitive material covering the optical path of the downhole spectroscopy device may be covered by one type of thickness of H₂S gas diffusion barrier or, alternatively, it may be covered by different thicknesses of H₂S gas diffusion barrier making the H₂S sensitive material capable of quantifying different concentrations of H₂S as mentioned above.

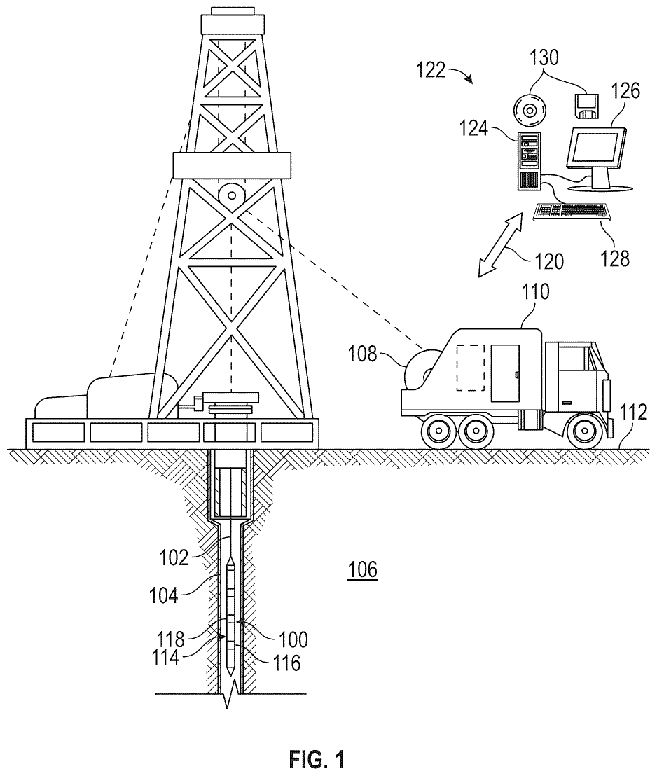

FIG. 1 is a schematic diagram of fluid sampling tool 100 on a conveyance 102. As illustrated, wellbore 104 may extend through subterranean formation 106. In examples, reservoir fluid may be contaminated with well fluid (e.g., drilling fluid) from wellbore 104. As described herein, the fluid sample may be analyzed to determine fluid contamination and other fluid properties of the reservoir fluid. As illustrated, wellbore 104 may extend through subterranean formation 106. While the wellbore 104 is shown extending generally vertically into subterranean formation 106, the principles described herein are also applicable to wellbores that extend at an angle through the subterranean formation 106, such as horizontal and slanted wellbores. For example, although FIG. 1 shows a vertical or low inclination angle well, high inclination angle or horizontal placement of the well and equipment is also possible. It should further be noted that while FIG. 1 generally depicts a land-based operation, those skilled in the art will readily recognize that the principles described herein are equally applicable to subsea operations that employ floating or sea-based platforms and rigs, without departing from the scope of the disclosure.

As illustrated, a hoist 108 may be used to run fluid sampling tool 100 into wellbore 104. Hoist 108 may be disposed on a vehicle 110. Hoist 108 may be used, for example, to raise and lower conveyance 102 in wellbore 104. While hoist 108 is shown on vehicle 110, it should be understood that conveyance 102 may alternatively be disposed from a hoist 108 that is installed at surface 112 instead of being located on vehicle 110. Fluid sampling tool 100 may be suspended in wellbore 104 on conveyance 102. Other conveyance types may be used for conveying fluid sampling tool 100 into wellbore 104, including coiled tubing and wired drill pipe, conventional drill pipe for example. Fluid sampling tool 100 may comprise a tool body 114, which may be elongated as shown on FIG. 1. Tool body 114 may be any suitable material, including without limitation titanium, stainless steel, alloys, plastic, any combinations thereof, and the like. Fluid sampling tool 100 may further include one or more sensors 116 for measuring properties of the fluid sample, reservoir fluid, wellbore 104, subterranean formation 106, or the like. In examples, fluid sampling tool 100 may also include a fluid analysis module 118, which may be operable to process information regarding fluid sample, as described below. The fluid sampling tool 100 may be used to collect fluid samples from subterranean formation 106 and may obtain and separately store different fluid samples from subterranean formation 106.

In examples, fluid analysis module 118 may comprise at least one sensor that may continuously monitor a reservoir fluid. Such sensors include optical sensors, acoustic sensors, electromagnetic sensors, conductivity sensors, resistivity sensors, a capacitance sensor, selective electrodes, density sensors, mass sensors, thermal sensors, chromatography sensors, viscosity sensors, bubble point sensors, fluid compressibility sensors, flow rate sensors. Sensors may measure a contrast between drilling fluid filtrate properties and formation fluid properties. Fluid analysis module 118 may be operable to derive properties and characterize the fluid sample. By way of example, fluid analysis module 118 may measure absorption, transmittance, or reflectance spectra and translate such measurements into component concentrations of the fluid sample, which may be lumped component concentrations, as described above. The fluid analysis module 118 may also measure gas-to-oil ratio, fluid composition, water cut, live fluid density, live fluid viscosity, formation pressure, and formation temperature. Fluid analysis module 118 may also be operable to determine fluid contamination of the fluid sample and may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, fluid analysis module 118 may include random access memory (RAM), one or more processing units, such as a central processing unit (CPU), or hardware or software control logic, ROM, and/or other types of nonvolatile memory.

Any suitable technique may be used for transmitting phase signals from the fluid sampling tool 100 to the surface 112. As illustrated, a communication link 120 (which may be wired or wireless, for example) may be provided that may transmit data from fluid sampling tool 100 to an information handling system 122 at surface 112. Information handling system 122 may include a processing unit 124, a monitor 126, an input device 128 (e.g., keyboard, mouse, etc.), and/or computer media 130 (e.g., optical disks, magnetic disks) that can store code representative of the methods described herein. The information handling system 122 may act as a data acquisition system and possibly a data processing system that analyzes information from fluid sampling tool 100. For example, information handling system 122 may process the information from fluid sampling tool 100 for determination of fluid contamination. The information handling system 122 may also determine additional properties of the fluid sample (or reservoir fluid), such as component concentrations, pressure-volume-temperature properties (e.g., bubble point, phase envelop prediction, etc.) based on the fluid characterization. This processing may occur at surface 112 in real-time. Alternatively, the processing may occur downhole hole or at surface 112 or another location after recovery of fluid sampling tool 100 from wellbore 104. Alternatively, the processing may be performed by an information handling system in wellbore 104, such as fluid analysis module 118. The resultant fluid contamination and fluid properties may then be transmitted to surface 112, for example, in real-time.

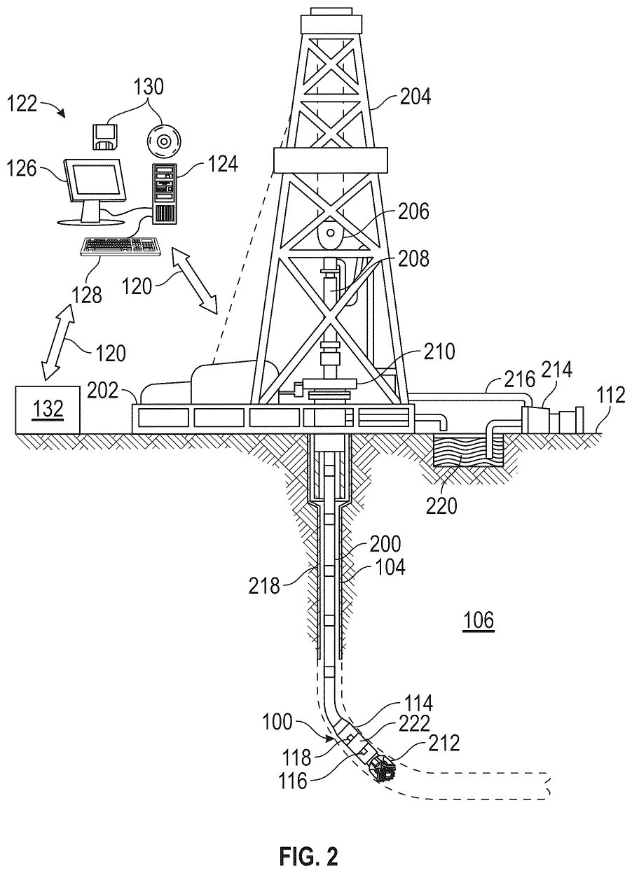

Referring now to FIG. 2, a schematic diagram illustrates a fluid sampling tool 100 disposed on a drill string 200 in a drilling operation. Fluid sampling tool 100 may be used to obtain a fluid sample, for example, a fluid sample of a reservoir fluid from subterranean formation 106. The reservoir fluid may be contaminated with well fluid (e.g., drilling fluid) from wellbore 104. As described herein, the fluid sample may be analyzed to determine fluid contamination and other fluid properties of the reservoir fluid. As illustrated, a wellbore 104 may extend through subterranean formation 106. While the wellbore 104 is shown extending generally vertically into the subterranean formation 106, the principles described herein are also applicable to wellbores that extend at an angle through the subterranean formation 106, such as horizontal and slanted wellbores. For example, although FIG. 2 shows a vertical or low inclination angle well, high inclination angle or horizontal placement of the well and equipment is also possible. It should further be noted that while FIG. 2 generally depicts a land-based operation, those skilled in the art will readily recognize that the principles described herein are equally applicable to subsea operations that employ floating or sea-based platforms and rigs, without departing from the scope of the disclosure.

As illustrated, a drilling platform 202 may support a derrick 204 having a traveling block 206 for raising and lowering drill string 200. Drill string 200 may include, but is not limited to, drill pipe and coiled tubing, as generally known to those skilled in the art. A kelly 208 may support drill string 200 as it may be lowered through a rotary table 210. A drill bit 212 may be attached to the distal end of drill string 200 and may be driven either by a downhole motor and/or via rotation of drill string 200 from the surface 112. Without limitation, drill bit 212 may comprise roller cone bits, PDC bits, natural diamond bits, any hole openers, reamers, coring bits, and the like. As drill bit 212 rotates, it may create and extend wellbore 104 that penetrates various subterranean formations 106. A pump 214 may circulate drilling fluid through a feed pipe 216 to kelly 208, downhole through the interior of drill string 200, through orifices in drill bit 212, back to surface 112 via annulus 218 surrounding drill string 200, and into a retention pit 220.

Drill bit 212 may be just one piece of a downhole assembly that may include one or more drill collars 222 and fluid sampling tool 100. Fluid sampling tool 100, which may be built into the drill collars 222 may gather measurements and fluid samples as described herein. One or more of the drill collars 222 may form a tool body 114, which may be elongated as shown on FIG. 2. Tool body 114 may be any suitable material, including without limitation titanium, stainless steel, alloys, plastic, any combinations thereof, and the like. Fluid sampling tool 100 may be similar in configuration and operation to fluid sampling tool 100 shown on FIG. 1 except that FIG. 2 shows fluid sampling tool 100 disposed on drill string 200. Alternatively, fluid sampling tool 100 may be lowered into the wellbore after drilling operations on a wireline.

Fluid sampling tool 100 may further include one or more sensors 116 for measuring properties of the fluid sample reservoir fluid, wellbore 104, subterranean formation 106, or the like. The properties of the fluid are measured as the fluid passes from the formation through the tool and into either the wellbore or a sample container. As fluid is flushed in the near wellbore region by the mechanical pump, the fluid that passes through the tool generally reduces its drilling fluid filtrate content, and generally increases its formation fluid content. The fluid sampling tool 100 may be used to collect a fluid sample from subterranean formation 106 when the filtrate content has been determined to be sufficiently low. Sufficiently low depends on the purpose of sampling. For some laboratory testing, below 10% drilling fluid contamination is sufficiently low, while for other testing, below 1% drilling fluid filtrate contamination is sufficiently low. Sufficiently low also depends on the nature of the formation fluid such that lower requirements are generally needed, the lighter the oil as designated with either a higher GOR or a higher API gravity. Sufficiently low also depends on the rate of cleanup in a cost benefit analysis since longer pump out times utilized to incrementally reduce the contamination levels may have prohibitively large costs. As previously described, the fluid sample may comprise a reservoir fluid, which may be contaminated with a drilling fluid or drilling fluid filtrate. Fluid sampling tool 100 may obtain and separately store different fluid samples from subterranean formation 106 with fluid analysis module 118. Fluid analysis module 118 may operate and function in the same manner as described above. However, storing the fluid samples in fluid sampling tool 100 may be based on the determination of the fluid contamination. For example, if the fluid contamination exceeds a tolerance, then the fluid sample may not be stored. If the fluid contamination is within a tolerance, then the fluid sample may be stored in fluid sampling tool 100.

As previously described, information from fluid sampling tool 100 may be transmitted to an information handling system 122, which may be located at surface 112. As illustrated, communication link 120 (which may be wired or wireless, for example) may be provided that may transmit data from fluid sampling tool 100 to an information handling system 122 at surface 112. Information handling system 122 may include a processing unit 124, a monitor 126, an input device 128 (e.g., keyboard, mouse, etc.), and/or computer media 130 (e.g., optical disks, magnetic disks) that may store code representative of the methods described herein. In addition to, or in place of processing at surface 112, processing may occur downhole (e.g., fluid analysis module 118). In examples, information handling system 122 may perform computations to estimate clean fluid composition.

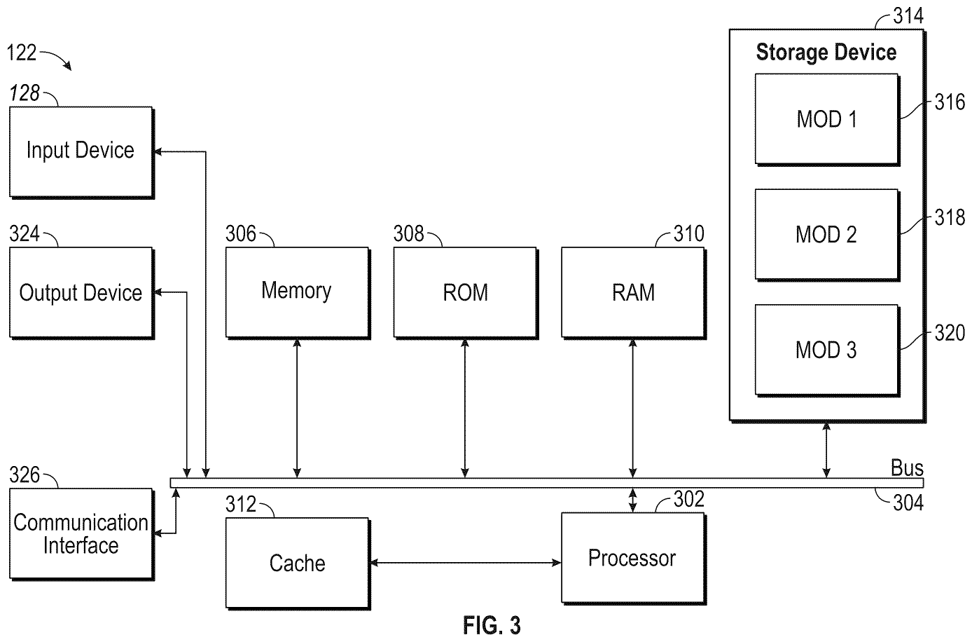

FIG. 3 illustrates information handling system 122 which may be employed to perform various blocks, methods, and techniques disclosed herein. As illustrated, information handling system 122 includes a processing unit (CPU or processor) 302 and a system bus 304 that couples various system components including system memory 306 such as read only memory (ROM) 308 and random-access memory (RAM) 310 to processor 302. Processors disclosed herein may all be forms of this processor 302. Information handling system 122 may include a cache 312 of high-speed memory connected directly with, in close proximity to, or integrated as part of processor 302. Information handling system 122 copies data from system memory 306 and/or storage device 314 to cache 312 for quick access by processor 302. In this way, cache 312 provides a performance boost that avoids processor 302 delays while waiting for data. These and other modules may control or be configured to control processor 302 to perform various operations or actions. Other system memory 306 may be available for use as well. System memory 306 may include multiple different types of memory with different performance characteristics. It may be appreciated that the disclosure may operate on information handling system 122 with more than one processor 302 or on a group or cluster of computing devices networked together to provide greater processing capability. Processor 302 may include any general-purpose processor and a hardware module or software module, such as first module 316, second module 318, and third module 320 stored in storage device 314, configured to control processor 302 as well as a special-purpose processor where software instructions are incorporated into processor 302. Processor 302 may be a self-contained computing system, containing multiple cores or processors, a bus, memory controller, cache, etc. A multi-core processor may be symmetric or asymmetric. Processor 302 may include multiple processors, such as a system having multiple, physically separate processors in different sockets, or a system having multiple processor cores on a single physical chip. Similarly, processor 302 may include multiple distributed processors located in multiple separate computing devices but working together such as via a communications network. Multiple processors or processor cores may share resources such as system memory 306 or cache 312 or may operate using independent resources. Processor 302 may include one or more state machines, an application specific integrated circuit (ASIC), or a programmable gate array (PGA) including a field PGA (FPGA).

Each individual component discussed above may be coupled to system bus 304, which may connect each and every individual component to each other. System bus 304 may be any of several types of bus structures including a memory bus or memory controller, a peripheral bus, and a local bus using any of a variety of bus architectures. A basic input/output (BIOS) stored in ROM 308 or the like, may provide the basic routine that helps to transfer information between elements within information handling system 122, such as during start-up. Information handling system 122 further includes storage devices 314 or computer-readable storage media such as a hard disk drive, a magnetic disk drive, an optical disk drive, tape drive, solid-state drive, RAM drive, removable storage devices, a redundant array of inexpensive disks (RAID), hybrid storage device, or the like. Storage device 314 may include software modules 316, 318, and 320 for controlling processor 302. Information handling system 122 may include other hardware or software modules. Storage device 314 is connected to the system bus 304 by a drive interface. The drives and the associated computer-readable storage devices provide nonvolatile storage of computer-readable instructions, data structures, program modules and other data for information handling system 122. In one aspect, a hardware module that performs a particular function includes the software component stored in a tangible computer-readable storage device in connection with the necessary hardware components, such as processor 302, system bus 304, and so forth, to carry out a particular function. In another aspect, the system may use a processor and computer-readable storage device to store instructions which, when executed by the processor, cause the processor to perform operations, a method or other specific actions. The basic components and appropriate variations may be modified depending on the type of device, such as whether information handling system 122 is a small, handheld computing device, a desktop computer, or a computer server. When processor 302 executes instructions to perform “operations”, processor 302 may perform the operations directly and/or facilitate, direct, or cooperate with another device or component to perform the operations.

As illustrated, information handling system 122 employs storage device 314, which may be a hard disk or other types of computer-readable storage devices which may store data that are accessible by a computer, such as magnetic cassettes, flash memory cards, digital versatile disks (DVDs), cartridges, random access memories (RAMs) 310, read only memory (ROM) 308, a cable containing a bit stream and the like, may also be used in the exemplary operating environment. Tangible computer-readable storage media, computer-readable storage devices, or computer-readable memory devices, expressly exclude media such as transitory waves, energy, carrier signals, EM waves, and signals per se.

To enable user interaction with information handling system 122, an input device 128 represents any number of input mechanisms, such as a microphone for speech, a touch-sensitive screen for gesture or graphical input, keyboard, mouse, motion input, speech and so forth. Additionally, input device 128 may receive acoustic or EM measurements from fluid sampling tool 100 (e.g., referring to FIGS. 1 and 2), discussed above. An output device 324 may also be one or more of a number of output mechanisms known to those of skill in the art. In some instances, multimodal systems enable a user to provide multiple types of input to communicate with information handling system 122. Communications interface 326 generally governs and manages the user input and system output. There is no restriction on operating on any particular hardware arrangement and therefore the basic hardware depicted may easily be substituted for improved hardware or firmware arrangements as they are developed.

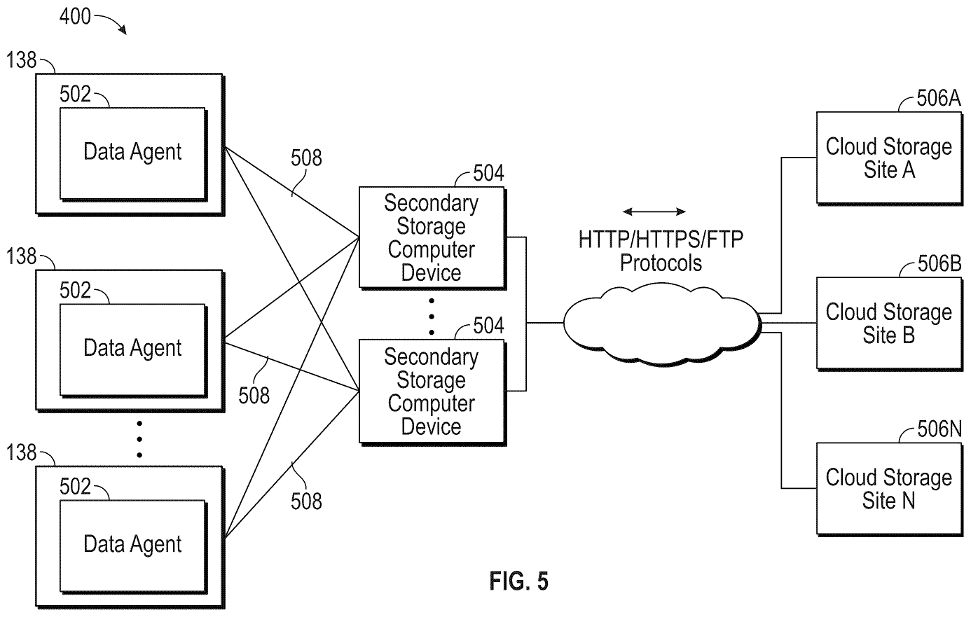

As illustrated, each individual component described above is depicted and disclosed as individual functional blocks. The functions these blocks represent may be provided through the use of either shared or dedicated hardware, including, but not limited to, hardware capable of executing software and hardware, such as a processor 302, that is purpose-built to operate as an equivalent to software executing on a general-purpose processor. For example, the functions of one or more processors presented in FIG. 5 may be provided by a single shared processor or multiple processors. (Use of the term “processor” should not be construed to refer exclusively to hardware capable of executing software.) Illustrative embodiments may include microprocessor and/or digital signal processor (DSP) hardware, read-only memory (ROM) 308 for storing software performing the operations described below, and random-access memory (RAM) 310 for storing results. Very large-scale integration (VLSI) hardware embodiments, as well as custom VLSI circuitry in combination with a general-purpose DSP circuit, may also be provided.

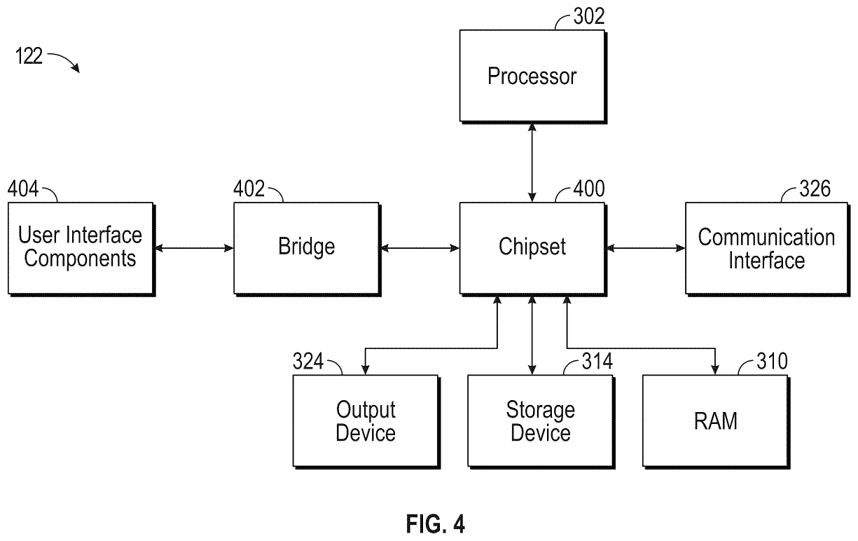

FIG. 4 illustrates an example information handling system 122 having a chipset architecture for information handling system 122 that may be used in executing the described method and generating and displaying a graphical user interface (GUI). Information handling system 122 is an example of computer hardware, software, and firmware that may be used to implement the disclosed technology. Information handling system 122 may include a processor 302, representative of any number of physically and/or logically distinct resources capable of executing software, firmware, and hardware configured to perform identified computations. Processor 302 may communicate with a chipset 400, discussed below, that may control input to and output from processor 302. In this example, chipset 400 outputs information to output device 324, such as a display, and may read and write information to storage device 314, which may include, for example, magnetic media, and solid-state media. Chipset 400 may also read data from and write data to RAM 310. Bridge 402 for interfacing with a variety of user interface components 404 may be provided for interfacing with chipset 400. Such user interface components 404 may include a keyboard, a microphone, touch detection and processing circuitry, a pointing device, such as a mouse, and so on. In general, inputs to information handling system 122 may come from any of a variety of sources, machine generated and/or human generated.

Chipset 400 may also interface with one or more communication interfaces 326 that may have different physical interfaces. Such communication interfaces may include interfaces for wired and wireless local area networks, for broadband wireless networks, as well as personal area networks. Some applications of the methods for generating, displaying, and using the GUI disclosed herein may include receiving ordered datasets over the physical interface or be generated by the machine itself by processor 302 analyzing data stored in storage device 314 or RAM 310. Further, information handling system 122 receives inputs from a user via user interface components 404 and executes appropriate functions, such as browsing functions by interpreting these inputs using processor 302.

In examples, information handling system 122 may also include tangible and/or non-transitory computer-readable storage devices for carrying or having computer-executable instructions or data structures stored thereon. Such tangible computer-readable storage devices may be any available device that may be accessed by a general purpose or special purpose computer, including the functional design of any special purpose processor as described above. By way of example, and not limitation, such tangible computer-readable devices may include RAM, ROM, EEPROM, CD-ROM or other optical disk storage, magnetic disk storage or other magnetic storage devices, or any other device which may be used to carry or store program code in the form of computer-executable instructions, data structures, or processor chip design. When information or instructions are provided via a network, or another communications connection (either hardwired, wireless, or combination thereof), to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general-purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing blocks of the methods disclosed herein. The particular sequence of such executable instructions or associated data structures represents examples of corresponding acts for implementing the functions described in such blocks.

In additional examples, methods may be practiced in network computing environments with many types of computer system configurations, including personal computers, hand-held devices, multi-processor systems, microprocessor-based or programmable consumer electronics, network PCs, minicomputers, mainframe computers, and the like. Examples may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

FIG. 5 illustrates an example of one arrangement of resources on a computing network 500 that may employ the processes and techniques described herein, although many others are of course possible. As noted above, an information handling system 122, as part of their function, may utilize data, which includes files, databases, directories, metadata (e.g., access control list (ACLS) creation/edit dates associated with the data, etc.), and other data objects. The data on the information handling system 122 is typically a primary copy (e.g., a production copy). During a copy, backup, archive or other storage operation, information handling system 122 may send a copy of some data objects (or some components thereof) to a secondary storage computing device 504 by utilizing one or more data agents 502.

A data agent 502 may be a desktop application, website application, or any software-based application that is run on information handling system 122. As illustrated, information handling system 122 may be disposed at any rig site (e.g., referring to FIG. 1), off site location, core laboratory, repair and manufacturing center, and/or the like. In examples, data agent 502 may communicate with a secondary storage computing device 504 using communication protocol 508 in a wired or wireless system. Communication protocol 508 may function and operate as an input to a website application. In the website application, field data related to pre-and post-operations, generated DTCs, notes, and/or the like may be uploaded. Additionally, information handling system 122 may utilize communication protocol 508 to access processed measurements, operations with similar DTCs, troubleshooting findings, historical run data, and/or the like. This information is accessed from secondary storage computing device 504 by data agent 502, which is loaded on information handling system 122.

Secondary storage computing device 504 may operate and function to create secondary copies of primary data objects (or some components thereof) in various cloud storage sites 506A-N. Additionally, secondary storage computing device 504 may run determinative algorithms on data uploaded from one or more information handling systems 122, discussed further below. Communications between the secondary storage computing devices 504 and cloud storage sites 506A-N may utilize REST protocols (Representational state transfer interfaces) that satisfy basic C/R/U/D semantics (Create/Read/Update/Delete semantics), or other hypertext transfer protocol (“HTTP”)-based or file-transfer protocol (“FTP”)-based protocols (e.g., Simple Object Access Protocol).

In conjunction with creating secondary copies in cloud storage sites 506A-N, the secondary storage computing device 504 may also perform local content indexing and/or local object-level, sub-object-level or block-level deduplication when performing storage operations involving various cloud storage sites 506A-N. Cloud storage sites 506A-N may further record and maintain, EM logs, map DTC codes, store repair and maintenance data, store operational data, and/or provide outputs from determinative algorithms that are located in cloud storage sites 506A-N. In a non-limiting example, this type of network may be utilized as a platform to store, backup, analyze, import, perform extract, transform and load (“ETL”) processes, mathematically process, apply machine learning models, and augment data sets.

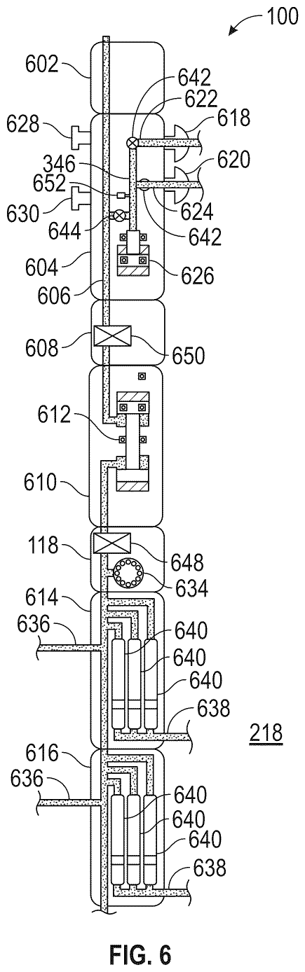

FIG. 6 is a schematic of fluid sampling tool 100. In some embodiments, fluid sampling tool 100 includes a power telemetry section 602 through which the tool communicates with other actuators and sensors 116 in drill string 200 or conveyance 102 (e.g., referring to FIGS. 1 and 2), the drill string's telemetry section 602, and/or directly with a surface telemetry system (not illustrated). In examples, power telemetry section 602 may also be a port through which the various actuators (e.g., valves) and sensors (e.g., temperature and pressure sensors) in fluid sampling tool 100 may be controlled and monitored. In examples, power telemetry section 602 includes a computer that exercises the control and monitoring function. In one embodiment, the control and monitoring function is performed by a computer in another part of the drill string or wireline tool (not shown) or by information handling system 122 on surface 112 (e.g., referring to FIGS. 1 and 2).

In examples, fluid sampling tool 100 includes a dual probe section 604, which extracts fluid from the reservoir and delivers it to fluid passageway 606 that extends from one end of fluid sampling tool 100 to the other. Without limitation, dual probe section 604 includes two probes 618, 620 which may extend from fluid sampling tool 100 and press against the inner wall of wellbore 104 (e.g., referring to FIG. 1). Probe channels 622, 624 may connect probes 618, 620 to fluid passageway 606. The high-volume bidirectional pump 612 may be used to pump fluids from the reservoir, through probe channels 622, 624 and to fluid passageway 606. The high-volume bidirectional pump 612 may contain from 100 cm³ to 1000 cm³, from 200 cm³ to 800 cm³, from 300 cm³ to 700 cm³, or any number in between. Alternatively, a low volume pump 626 may be used for this purpose. The low-volume pump 626 may contain from 10 cm³ to 400 cm³, from 20 cm³ to 300 cm³, from 30 cm³ to 200 cm³, from 50 cm³ to 100 cm³, or any number in between. Two standoffs or stabilizers 628, 630 hold fluid sampling tool 100 in place as probes 618, 620 press against the wall of wellbore 104. In examples, probes 618, 620 and stabilizers 628, 630 may be retracted when fluid sampling tool 100 may be in motion and probes 618, 620 and stabilizers 628, 630 may be extended to sample the formation fluids at any suitable location or sampling zone in wellbore 104. Other probe sections include focused sampling probes, oval probes, or packers (not shown).

In examples, fluid passageway 606 may be connected to other tools disposed on drill string 200 or conveyance 102 (e.g., referring to FIGS. 1 and 2). In examples, fluid sampling tool 100 may also include a quartz gauge section 608, which may include sensors 116 to allow measurement of properties, such as temperature and pressure, of fluid in fluid passageway 606. Additionally, fluid sampling tool 100 may include a flow-control pump-out section 610, which may include a high-volume bidirectional pump 612 for pumping fluid through fluid passageway 606. In examples, fluid sampling tool 100 may include two multi-chamber sections 614, 616, referred to collectively as multi-chamber sections 614, 616 or individually as first multi-chamber section 614 and second multi-chamber section 616, respectively.

In examples, multi-chamber sections 614, 616 may be separated from flow-control pump-out section 610 by sensor section 632, which may house at least one non-optical fluid sensor 648 and/or at least optical measurement tool 634. It should be noted that non-optical fluid sensor 648 and optical measurement tool 634 may be disposed in any order on fluid passageway 606. Additionally, although depicted in sensor section 632, non-optical fluid sensor 648 and optical measurement tool 634 may be disposed along fluid passageway 606 at any suitable location within fluid sampling tool 100.

Non-optical fluid sensor 648 may be displaced within sensor section 632 in-line with fluid passageway 606 to be a “flow through” sensor. In alternate examples, non-optical fluid sensor 648 may be connected to fluid passageway 606 via an offshoot of fluid passageway 606. Without limitation, non-optical fluid sensor 648 may include but not limited to the density sensor, capacitance sensor, resistivity sensor, and/or any combinations thereof. In examples, non-optical fluid sensor 648 may operate and/or function to measure fluid properties of drilling fluid filtrate.

Optical measurement tool 634 may be displaced within sensor section 632 in-line with fluid passageway 606 to be a “flow through” sensor. In alternate examples, optical measurement tool 634 may be connected to fluid passageway 606 via an offshoot of fluid passageway 606. Without limitation, optical measurement tool 634 may include optical sensors, acoustic sensors, electromagnetic sensors, conductivity sensors, resistivity sensors, a capacitance sensor, selective electrodes, density sensors, mass sensors, thermal sensors, chromatography sensors, viscosity sensors, bubble point sensors, fluid compressibility sensors, flow rate sensors, microfluidic sensors, selective electrodes such as ion selective electrodes, and/or any combinations thereof. In example embodiments, optical measurement tool 634 may operate and/or function to measure drilling fluid filtrate as discussed further below.

Additionally, multi-chamber section 614, 616 may comprise access channel 636 and chamber access channel 638. Without limitation, access channel 636 and chamber access channel 638 may operate and function to either allow a solids-containing fluid (e.g., mud) disposed in wellbore 104 or provide a path for removing fluid from fluid sampling tool 100 into wellbore 104. As illustrated, multi-chamber section 614, 616 may comprise a plurality of chambers 640. Chambers 640 may be sampling chamber that may be used to sample wellbore fluids, formation fluids, and/or the like during measurement and sampling operations.

During downhole measurement operations, a pump out operation may be performed. A pump out may be an operation where at least a portion of a fluid which may contain solids—(e.g., drilling fluid, mud, filtrate etc.) may move through fluid sampling tool 100 until substantially increasing concentrations of formation fluids enter fluid sampling tool 100. For example, during pump out operations, probes 618, 620 may be pressed against the inner wall of wellbore 104 (e.g., referring to FIG. 1). Pressure may increase at probes 618, 620 due to compression against the formation 106 (e.g., referring to FIG. 1 or 2) exerting pressure on probes 618, 620. As pressure rises and reaches a predetermined pressure, valve 642 opens so as to close equalizer valve 644, thereby isolating fluid passageway 606 from annulus 218 (e.g., referring to FIG. 2). In this manner, valve 642 ensures that equalizer valve 644 closes only after probes 618, 620 has entered contact with mud cake (not illustrated) that is disposed against the inner wall of wellbore 104. In examples, as probes 618, 620 are pressed against the inner wall of wellbore 104, the pressure rises and closes the equalizer valve 644 in fluid passageway 606, thereby isolating fluid passageway 606 from the annulus 218. In this manner, the equalizer valve 644 in fluid passageway 606 may close before probes 618, 620 may have entered into contact with the mud cake that lines the inner wall of wellbore 104. Fluid passageway 606, now closed to annulus 218, is in fluid communication with low volume pump 626.

As low volume pump 626 is actuated, formation fluid may thus be drawn through probe channels 622, 624 and probes 618, 620. The movement of low volume pump 626 lowers the pressure in fluid passageway 606 to a pressure below the formation pressure, such that formation fluid is drawn through probe channels 622, 624 and probes 618, 620 and into fluid passageway 606. Probes 618, 620 serves as a seal to prevent annular fluids from entering fluid passageway 606. Such an operation as described may take place before, after, during or as part of a sampling operation.

With low volume pump 626 in its fully retracted position and formation fluid drawn into fluid passageway 606, the pressure will stabilize and enable pressure sensor 652 to sense and measure formation fluid pressure. The measured pressure is transmitted to information handling system 122 disposed on formation testing tool 100 and/or it may be transmitted to the surface via mud pulse telemetry or by any other conventional telemetry means to information handling system 122 disposed on surface 112.

During this interval, pressure sensor 652 may continuously monitor the pressure in fluid passageway 606 until the pressure stabilizes, or after a predetermined time interval. When the measured pressure stabilizes, or after a predetermined time interval, for example at 1800 psi, and is sensed by pressure sensor 652 the drawdown operation may be complete.

Next, high-volume bidirectional pump 612 activates and equalizer valve 644 is opened. This allows for formation fluid to move toward high-volume bidirectional pump 612 through fluid passageway 606. Formation fluid moves through fluid passageway 606 to sensor section 632. Once the drilling fluid filtrate has moved into sensor section 632, high-volume bidirectional pump 612 may stop. This may allow the drilling fluid filtrate to be measured by optical measurement tool 634 within sensor section 632. Without limitation, any suitable properties of the formation fluid may be measured utilizing an optical measurement tool.

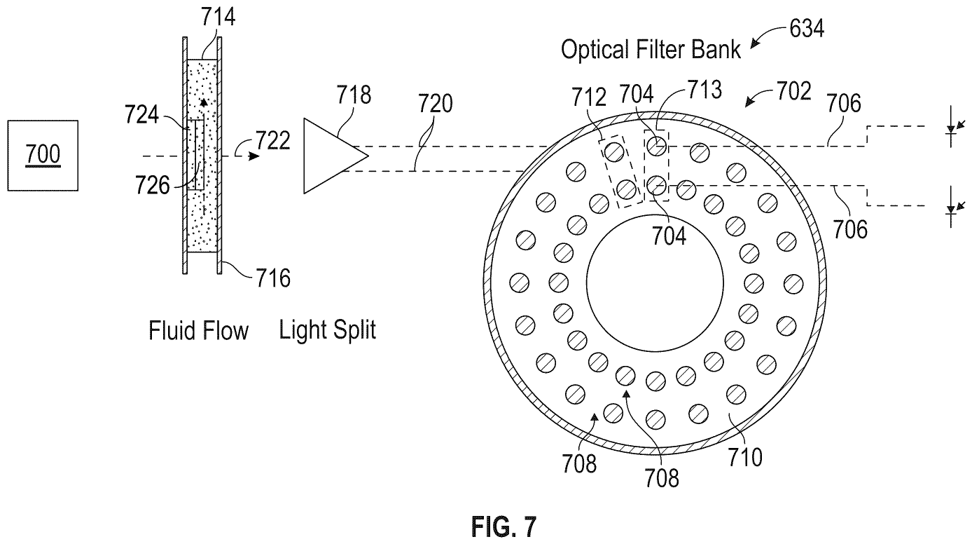

FIG. 7 depicts a hardware configuration of a dynamic subsurface optical measurement tool 634. It should be noted that a channel disclosed herein may be a measurement of the light transmittance through an optical filter. Optical measurement tool 634 may include a light source 700, a filter bank 702 comprising a plurality of optical filters 704 (measurement of the light transmittance through an optical filter 704 is called a channel 706) configured as two rings 708 on optical plate 710, within a channel pair 712 on each azimuth. It should be noted that each channel 706 may be designed, based on the construction of each channel 706 respective to optical filter 704, to measure different properties of fluid sample 714. During the rotation of optical plate 710, the two optical filters 704 on a channel pair 712 may be synchronized spatially or in time to measure substantially the same fluid sample 714 in viewing area 716. As discussed below, and illustrated in FIG. 7, an active channel pair 713 is a channel pair 712 in which optical measurements are being taken to form one or more channels 706. In some embodiments, channel pairs 712 may be near synchronized such that channel pairs 712 have a sufficient probability of observing the same phase, i.e., better than 10% but more desirably more than 50% and yet more desirably more than 80%. In other embodiments, more than two channels 706 may be sufficiently synchronized according to a desired probability of observing a single phase in time or space. A velocity calculation of the fluid phase specific velocities may be used to aid synchronization over longer distances, or time. Alternatively, distribution calculations, or autocorrelation calculations may be used to improve synchronization over longer distances or time. If the channels are sufficiently close in distance or time, the channel signals may not need additional efforts of synchronization. During measurement operations, fluid samples 714 (which is formation fluid from fluid passageway 606) may flow through viewing area 716 as a non-limiting example constructed by a set of windows or other transparent region of the flow path. Windows of viewing area 716 may be sapphire windows. Alternatively, the viewing region or viewing area 716 might not be transparent to visible light but rather to the form of energy used to measure the fluid characteristics for a given sensor. As such a viewing region or viewing area 716 for an acoustic sensor would ideally have a low acoustic impedance even if it is not transparent to visible light. Alternatively, the viewing region or viewing area 716 may be transparent (i.e., pass energy with low attenuation) to infrared light, or magnetic fields instead of visible light. In some embodiments for some sensors, viewing area 716 is more generally a measurement region or area as is the case with some phase behavior sensors or some density sensors. In examples, viewing area 716 may be at least a part of fluid passageway 606 and/or a branch of fluid passageway 606. In one nonlimiting embodiment, light 722 absorbed by fluid sample 714 may be split into at least two ray paths 720, through a prism 718. Split light rays 720 may be measured by detectors, not shown, as they pass through channel pair 712 separately. Filter bank 702 may rotate to another channel pair 712 after the measurement of each channel 706 from channel pair 712 and may dynamically gather an optical spectra measurement of all channels after a full sampling channel rotation. It should be noted, the methods disclosed herein may not be limited in simultaneous measurements of a channel pair 712 (two optical filters 704 and their respective channel 706) but may also apply to cases with one or more optical filters 704 or filter banks 702, at least one channel 706, or, alternatively, two or more channels 706.

As described, optical measurement tool 634 may be used in a downhole environment to perform measurements on fluid samples 714 to determine if a target analyte may or may not be present within fluid samples 714. In examples, a target analyte may by hydrogen sulfide (H₂S), carbon dioxide (CO₂), mercury (Hg), any other corrosive analyte, or any combination thereof. Currently, there is no downhole, real time, in-situ, measurement of high concentration of H₂S in gas phase or in liquid phase, for example. A potential solution for a downhole, real time, H₂S sensor would be to use a H₂S gas diffusion barrier 726 such as beryllium oxide that may control the diffusion rate of H₂S from fluid samples 714 in the flow line to a thin metal film 724 or semiconductor that reacts with the H₂S gas, which may be probed via optical or electrical measurement methods. Thin metal film 724 may be tin oxide alloyed doped with silver, chromium oxide alloyed doped with silver, indium tin oxide alloyed doped with silver, or any combination thereof.

Thin metal film 724 may be deposited on viewing area 716 or more generally on a substrate in a measurement region, wherein light 722 can go through (referring to FIG. 7). For example, thin metal film 724 may change at least one of its physical properties upon exposure to the target analyte. The physical properties of thin metal film 724 impacted by the target analyte may be any physical properties that can be measured including electrical properties (such as its resistance, capacitance, conductance, or inductance for example) and color. Thin metal film 724 such as tin oxide alloyed doped with silver, chromium oxide alloyed doped with silver, indium tin oxide alloyed doped with silver, or any combination thereof could react and undergo a color change upon exposure to the target analyte, which may be probed and/or analyzed with optical measurement tool 634, for example. Color change may take the form of darkening of thin metal film 724 but change may also extend to outside visible spectrum. For example, thin metal film 724 may change on the ultraviolet and/or infrared scale, a color change not viewable to the naked eye. The change of the physical property of thin metal film 724 may be linear as a function of the concentration of the target analyte it is exposed to.

There may be one or more zones of thin metal film 724 without any H₂S gas diffusion barrier 726 and one or more zones of thin metal film 724 covered by H₂S gas diffusion barrier 726 to slow down H₂S diffusion and its reaction with thin metal film 724. Further, H₂S gas diffusion barrier 726 may be applied in different concentration and/or thickness over thin metal film 724 for thin metal film 724 to be sensitive to different concentration of H₂S. In embodiments, thin metal film 724 may cover the optical path of light 722 only partially. Then, thin metal film 724 covering the optical path of light 722 may be covered by one type of thickness of H₂S gas diffusion barrier 726 or, alternatively, it may be covered by different thicknesses of H₂S gas diffusion barrier 726 making thin metal film 724 capable of quantifying different concentrations of H₂S as mentioned above. Thin metal film 724 may be covered by H₂S gas diffusion barrier 726 in a pattern with different thicknesses or layers.

Further, the change of physical property may be reversible naturally such that as the concentration of the target analyte decreases from one sampling zone to another sampling zone, the physical property of thin metal film 724 changes accordingly. Alternatively, thin metal film 724 may be reset upon annealing thin metal film 724 to high temperatures such as 300° C., 400° C., 500° C., 750° C., 1000° C., or above. Annealing thin metal film 724 may be performed by electrical resistance heating, induction heating, electromagnetic heating, or any combination, for example.

Alternatively, thin metal film 724 may be reset upon exposure to a gas such as oxygen. In embodiments, thin metal film 724 may be reset upon exposure to a gas such as oxygen at high temperatures such as 200° C., 300° C., 400° C., 500° C., 750° C., 1000° C., or above. Oxygen or any other gas that may be used to reset thin metal film 724 to a physical property corresponding to thin metal film 724 without any target analyte may be transported downhole in one or more sampling chambers 640.

Thin metal film 724 may comprise at least in part a transition metal. In examples, thin metal film 724 may be selected from at least one metal selected from the list of metals comprising tin oxide alloyed doped with silver, chromium oxide alloyed doped with silver, indium tin oxide alloyed doped with silver, or any combination thereof as an indicator for H₂S gas or any target analyte. Additionally, thin metal film 724 may be deposited and/or disposed on a substrate such as calcium fluoride, glass, pure optical glass such as BK7, SiO₂, Al₂O₃, Sapphire, or Magnesium Fluoride, for example. Further, the thickness of thin metal film 724 may be any thickness as long as infrared can be transmitted such as less than 300 nm thick, less than 150 nm thick, less than 100 nm thick, less than 50 nm thick, less than 25 nm thick, less than 10 nm thick, less than 5 nm thick, or less than 3 nm thick, for example. Any thicker, and thin metal film 724 may be opaque and no light intensity may be observed via transmission. In example, the thickness of thin metal film 724 may be utilized to accentuate color change. If thin metal film 724 is a metal oxide, metal oxides are transparent and therefore no upper limit is necessary for their thickness. Upon surface adsorption or chemical reaction, these metals may have their optical and electrical properties change. Optical changes to thin metal film 724 may be viewed through viewing area 716.

For viewing area 716, thin metal film 724 or a semiconductor, which may comprise at least in part the metals described above, may be coated onto viewing area 716. In other examples, thin metal film 724 may be disposed on a substrate within viewing area 716 but not deposited on viewing area 716. If thin metal film 724 is a single film, the optical transmission intensity may be measured directly, indicating the presence of H₂S. Further examples may comprise one or more array and/or patterns of deposited materials with different H₂S concentration susceptibility to be selectively probed and/or analyzed with optical measurement tool 634. The patterns or arrays may be probed through the use of a mask to block light 722, polarize light 722, or polarization masks to measure specific regions of fluid sample 714. The patterns may be a four-quadrant circle, or a unique pattern generated with a mask. The use of a mask may allow for selecting from one or more thin metal films 724 during measurement operations, which may extend the life of each thin metal film 724. H₂S gas diffusion barrier 726 may be deposited on thin metal film 724 as described above including a homogenous thin film or pattern of H₂S gas diffusion barrier 726 with different thicknesses or concentration on thin metal film 724.

H₂S gas diffusion barrier 726 includes any gas diffusion barrier capable of slowing down the diffusion of H₂S including beryllium oxide, thin dielectric, semiconductor thin film with a lattice constant smaller than H₂S, and any combination thereof. H₂S gas diffusion barrier 726 may be from about 1 nm to about 1000 nm thick, from about 5 nm to about 500 nm thick, from about 10 nm to about 250 nm thick, from about 20 nm to about 200 nm thick, from about 25 nm to about 100 nm thick, or from about 50 nm to about 75 nm thick, for example.

For example, a plurality of H₂S gas diffusion barrier 726 deposited on a plurality of thin metal films 724 may be disposed in an array that may be linear, stacked, and/or a combination thereof. The mask may cover all, one, or a plurality of thin metal films 724 in the array. This may allow for the selection of specific thin metal films 724, which may extend the life of each thin metal film 724. This may be performed by having each thin metal film 724 having a different saturation point. At least one thin metal film 724 may have a low saturation point, another a medium saturation point, and a third may have a high saturation point. At any depth within wellbore 104, the concentration of a target analyte may be generally known and/or perceived. In areas in which there may be low concentrations of target analyte, the mask may uncover thin metal films 724 that have a low saturation point and cover the other thin metal films 724 with higher saturation points. At another depth, in which concentrations of the target analyte may be perceived to be high, the mask may uncover thin metal films 724 with high saturation points and cover thin metal films 724 with lower saturation points. This may allow each thin metal film 724 to last longer as they may not become saturated quickly.

Alternatively, following the reaction with H₂S, the conductivity/resistivity of thin metal film 724 may be probed through electrical measurements. As noted above, thin metal film 724 may comprise at least in part conducting or semiconducting materials. In other examples, a substrate upon which thin metal film 724 may be disposed may comprise at least in part conducting or semiconducting material. For an electrical sensor, a single coating or array of materials with different H₂S concentration susceptibility may be fabricated on individual circuits. Different thicknesses of thin metal film 724 may be used for different concentrations of H₂S. The quantity of the reactive metal (such as tin oxide alloyed doped with silver, chromium oxide alloyed doped with silver, indium tin oxide alloyed doped with silver, or any combination thereof) on thin metal film 724 may be set to target different ranges of concentrations of H₂S. The target range of concentration of H₂S may span from the range of concentrations of H₂S around the 1^{st} National Association of Corrosion Engineers (NACE) limit (corresponding to a partial pressure of 0.05 psi) all the way to a range of concentrations of H₂S around a partial pressure of H₂S above 3.0 psi. The electrical properties may then be probed via conductance, resistance, capacitance, or inductance measurements. Optical measurements and electrical measurements described above may also be utilized to identify mercury (Hg), carbon dioxide (CO₂), and any other reactive downhole analyte.

Optical measurement and electrical measurements may collect data regarding the possibility of H₂S, or other analyte within fluid sample 714. Analysis of collected data may occur at various locations in a system or at various steps in a method in accordance with the present disclosure. For example, processing of the collected data may occur at any suitable location including, without limitation, at fluid analysis module 118 and/or information handling system 122.

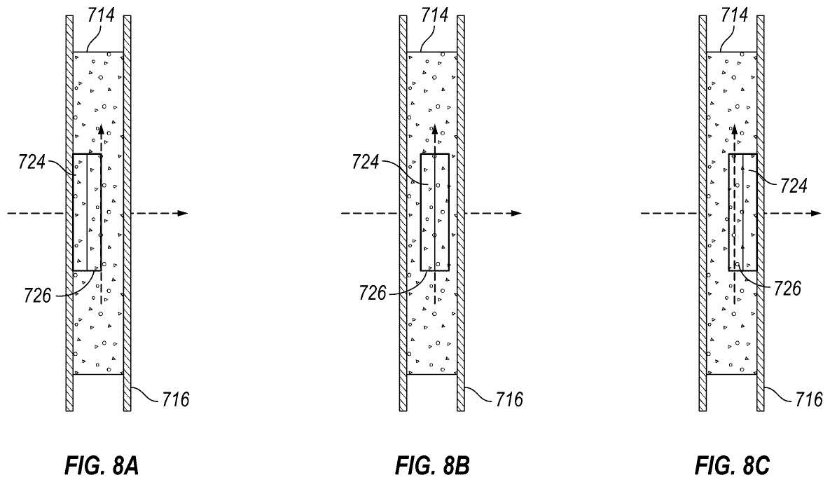

FIG. 8A is another view of the design of H₂S gas diffusion barrier 726 deposited over thin metal film 724 deposited on a substrate in viewing area 716 of FIG. 7. The light of optical path of light 722 goes through thin metal film 724 first and then H₂S gas diffusion barrier 726. H₂S gas diffusion barrier 726 may cover the entirety of thin metal film 724 including the three sides exposed to fluid sample 714 (not shown) or cover only the side facing the center of the flow line as shown.

FIG. 8B depicts another design of H₂S gas diffusion barrier 726 deposited over thin metal film 724 located in the center of fluid passageway 606 (referring to FIG. 6). In this design, H₂S gas diffusion barrier 726 covers the entirety of thin metal film 724 including the four sides exposed to fluid sample 714. While optical path of light 722 goes from left to right in FIG. 8B, it may be oriented in the other direction. As illustrated in FIG. 8B, the geometry of H₂S gas diffusion barrier 726 deposited over thin metal film 724 is designed to allow fluid flow around the sensing element while channeling optical energy along optical path of light 722. This geometry may comprise at least two factors that may be optimized to any given analysis. These factors may comprise optical path of light 722 and flow dynamics.

FIG. 8C depicts another design of H₂S gas diffusion barrier 726 deposited over thin metal film 724 deposited on a substrate in viewing area 716 wherein light of optical path of light 722 goes through H₂S gas diffusion barrier 726 first and then thin metal film 724. H₂S gas diffusion barrier 726 may cover the entirety of thin metal film 724 including the three sides exposed to fluid sample 714 (not shown) or cover only the side facing the center of the flow line as shown.

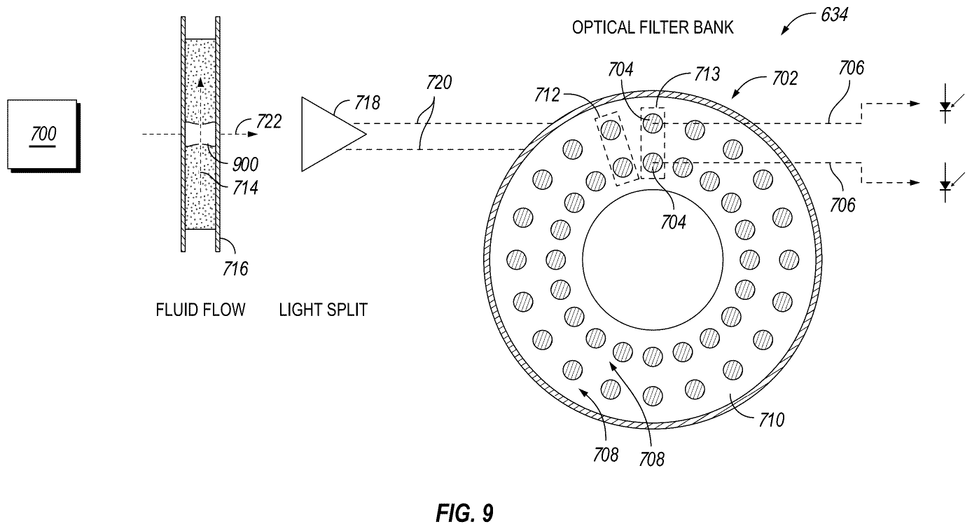

FIG. 9 depicts an alternative bridge design 900 with H₂S gas diffusion barrier 726 (not shown in FIG. 9) deposited on thin metal films 724 (not shown) in the dynamic subsurface optical measurement tool 634. As described in FIG. 7, optical measurement tool 634 may include light source 700, filter bank light modifier 702 comprising a plurality of optical filters 704 (measurement of the light transmittance through optical filter 704 is called channel 706) configured as two rings 708 on optical plate 710, within channel pair 712 on each azimuth. Bridge design 900 may be inserted into viewing area 716 and act as a conduit within the optical path of light 722 as illustrated in FIG. 9. In examples, bridge design 900 may be a permanent structure and in other examples, it may be removable. Bridge design 900 is coated with thin metal film 724 which is covered, at least partially, with gas diffusion barrier (not shown). As mentioned above, thin metal film 724 covering the optical path of light 722 may be covered by one type of thickness or homogeneous layer of H₂S gas diffusion barrier 726 or, alternatively, it may be covered by different thicknesses of H₂S gas diffusion barrier 726 making thin metal film capable of quantifying different concentrations of H₂S. Thin metal film 724 may be covered by H₂S gas diffusion barrier 726 in a pattern with different thicknesses or layers.

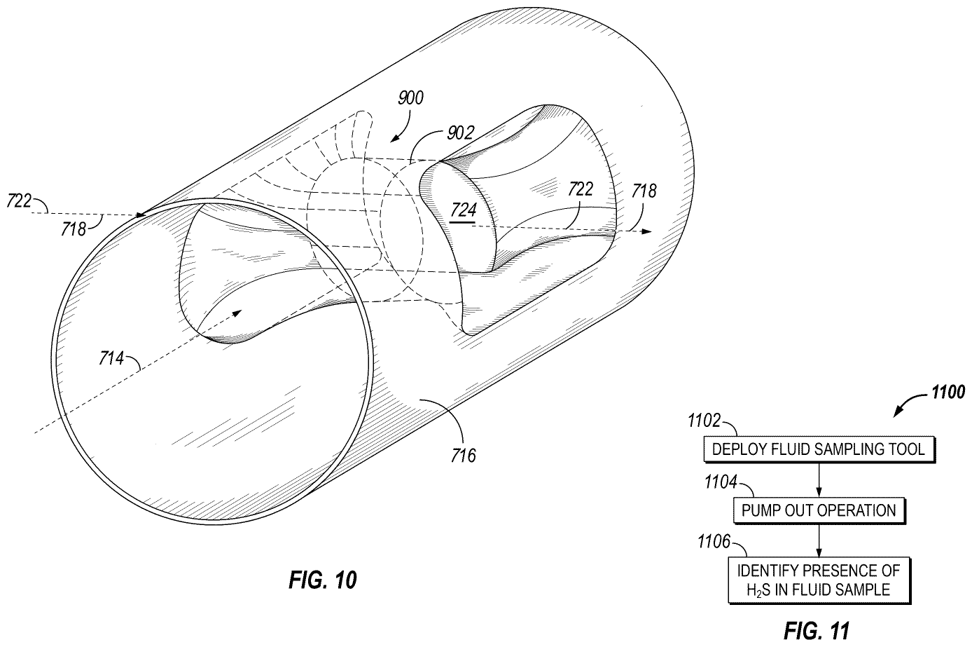

As illustrated in FIG. 10, bridge design 900 may comprise a structure 902, which includes at least one substrate, thin metal films 724, and optionally a mechanical support, that is connected on one side of the viewing area 716 and is further connected to a second side of the viewing area 716 along light 722. This may form a “window” between light source 700 and filter bank 702. Bridge design 900 may form a structure of any shape as to allow for the placement of a thin metal film 724 within the fluid sample 714 through viewing area 716. Thin metal film 724 may be bound to the surface, contained within or otherwise immobilized by a substrate and held in place by structure 902. Structure 902 may be a porous and permeable form such as but not limited to a filter disk at least partially hollowed out for which fluid may freely flow but the structure remains in a local position within the viewing area 716. H₂S gas diffusion barrier 726 (not shown) may be deposited onto thin metal film 724 as described above including a homogenous thin film or pattern of H₂S gas diffusion barrier 726 with different thicknesses or concentration on thin metal film 724.

As illustrated in FIG. 9 and FIG. 10, the geometry of bridge design 900 is designed to allow fluid flow around bridge design 900 while channeling optical energy along optical path of light 722. The geometry of bridge design 900 may comprise three factors that may be optimized to any given analysis. These factors may comprise optical path of light 722, flow dynamics, and substrate miscibility for which volume to surface area is a characteristic. For example, bridge design 900 may be soluble to a selected phase for which that selected phase permeates and absorbs into the substrate of structure 902. To help in absorption, bridge design 900 may have a large surface area to volume ratio in order to maximize the adsorption of compatible fluid. Additionally, geometry of the shape of bridge design 900 may be designed to optimize the transmission of optical energy. The geometry of bridge design 900 may also promote flow across bridge design 900 and prevent buildup of particles. These three competing features provide different optimal designs for different environments; however, a generic shape is shown in FIG. 10.

FIG. 11 illustrates a workflow 1100 to determine the presence of hydrogen sulfide (H₂S) within fluid sample 714 (e.g., referring to FIG. 7). For this disclosure, workflow 1100 may be at least in part processed and/or performed on information handling system 122 (e.g., referring to FIG. 1). As illustrated, workflow 1100 may begin in block 1102. In block 1102, fluid sampling tool 100 may be deployed downhole to one or more selected depths within wellbore 104 (e.g., referring to FIG. 1). As described previously, once conveyed at a selected depth or sampling zone within wellbore 104, dual probe section 604 of fluid sampling tool 100 may be pressed against the inner wall of wellbore 104. Workflow 1100 may proceed to block 1104, where a pump out operation may occur. In block 1104, fluid sampling tool 100 may be used to pump out a fluid sample from formation 106 to be measured and/or analyzed with optical spectroscopy, such as via optical measurement tool 634 and fluid analysis module 118 (e.g., referring to FIG. 1). Pump out is pursued to clean reservoir fluid extracted from formation 106. Once the fluid sample 714 is deemed representative of a clean reservoir fluid from formation 106 (i.e. drilling fluid filtrate represents less than 10 % of the reservoir fluid, for example), workflow 1100 may proceed to block 1106.

In block 1106, fluid analysis module 118 and/or information handling system 122 may be used to interpret the information gathered by the optical measurement tool 634 and identify the presence and concentration of H₂S within fluid sample 714 using the methods and systems described above. The presence and/or concentration of H₂S, if present, may then be relayed to surface to personnel who may view the measurements on information handling system 122. Depending upon the thickness of H₂S gas diffusion barrier 726 over thin metal film 724 (referring to FIGS. 7-10), the sensitivity may be tuned to the range of H₂S present at the selected depth of formation 106 or sampling zone. If similar reservoir fluids have never been analyzed, the operator may choose a wide range of thickness of H₂S gas diffusion barrier 726 to be sensitive to a wide range of downhole concentrations of H₂S. If similar reservoir fluids have already been analyzed (i.e. different wells believed to be connected to the same reservoir fluids have already been analyzed), specific thicknesses of H₂S gas diffusion barrier 726 may be chosen to cover thin metal film 724 to be most sensitive to the concentration of H₂S believed to be present downhole.

Currently, H₂S is tested downhole on test coupons disposed in a downhole tool. These test coupons change color based on the concentration of H₂S. However, the color change is not reversible, and they cannot be monitored continuously and in real time (in other words, these coupons are inspected after the job to determine the highest H₂S concentration these coupons have been exposed to). Discussed above are methods and systems that are an improvement over current technology. Specifically, methods and systems for measuring H₂S concentration in a fluid sample in real-time during measurement operations are discussed. The systems and methods for determining H₂S concentration in real time in a fluid sample may include any of the various features of the systems and methods disclosed herein, including one or more of the following statements.

Statement 1. A method comprising: conveying a fluid sampling tool into a wellbore wherein the fluid sampling tool comprises: at least one probe to fluidly connect the fluid sampling tool to a formation in the wellbore; and at least one passageway that passes through the at least one probe and into the fluid sampling tool; drawing a formation fluid from a first sampling zone, as a fluid sample, through the at least one probe and through the at least one passageway; passing the fluid sample over a H₂S gas diffusion barrier that controls H₂S diffusion from the fluid sample to a thin film deposited on a measurement region, wherein the measurement region is part of the at least one passageway; and analyzing the fluid sample in the fluid sampling tool for a target analyte.

Statement 2. The method of Statement 1, wherein the thin film comprises at least one metal selected from a group of metals consisting of tin oxide alloyed doped with silver, chromium oxide alloyed doped with silver, indium tin oxide alloyed doped with silver, and any combination thereof.

Statement 3. The method of Statement 1 or Statement 2, wherein the thin film has a change of at least one property when the H₂S comes into contact with the thin film.

Statement 4. The method of any one of Statements 1-3; wherein the change of the at least one property of the thin film is proportional to a concentration of the H₂S.

Statement 5. The method of any one of Statements 1-4, wherein the H₂S gas diffusion barrier is beryllium oxide.

Statement 6. The method of any one of Statements 1-5, wherein the thin film changes optical properties when the H₂S comes into contact with the thin film.

Statement 7. The method of any one of Statements 1-6, further comprising identifying a change in optical properties of the thin film with an optical measurement tool, wherein the change in optical properties is proportional to a concentration of the H₂S, and wherein the concentration of the H₂S is determined from a linear, multivariate, or non-linear calibration model.

Statement 8. The method of any one of Statements 1-7, wherein one or more electrical properties of the thin film changes when the H₂S contacts the thin film.

Statement 9. The method of any one of Statements 1-8, wherein the one or more electrical properties comprise conductance, resistance, or inductance.

Statement 10. The method of any one of Statements 1-9, further comprising passing the fluid sample over a plurality of thin films.

Statement 11. The method of any one of Statements 1-10, wherein the plurality of thin films is disposed in an array or a pattern.

Statement 12. The method of any one of Statements 1-11, wherein the H₂S gas diffusion barrier is a homogeneous layer over the thin film.

Statement 13. The method of any one of Statements 1-12, wherein the H₂S gas diffusion barrier covers at least partially the thin film with layers of different thicknesses.

Statement 14. The method of any one of Statements 1-13, further resetting the thin film after exposure to the H₂S by annealing the thin film under exposure to a gas transported downhole in a sample chamber.

Statement 15. A system comprising a fluid sampling tool comprising: at least one probe to fluidly connect the fluid sampling tool to a formation in a wellbore; at least one passageway that passes through the at least one probe and into the fluid sampling tool; a sensor section comprising a measurement region in fluid communication with the at least one passageway; and a thin film deposited on the measurement region, wherein the thin film changes at least one of its physical properties upon exposure to a hydrogen sulfide (H₂S) found in the at least one passageway and wherein the thin film is covered at least partially by a H₂S gas diffusion barrier.

Statement 16. The system of Statement 15, wherein the thin film comprises at least one metal selected from a group of metals consisting of tin oxide alloyed doped with silver, chromium oxide alloyed doped with silver, indium tin oxide alloyed doped with silver, and any combination thereof.

Statement 17. The system of Statement 15 or Statement 16, wherein the change of the at least one of the physical properties of the thin film is proportional to a concentration of the H₂S.

Statement 18. The system of any one of Statements 15-17, further comprising at least one sampling chamber filled with a gas used to reset the thin film after exposure to the H₂S.

Statement 19. The system of any one of Statements 15-18, further comprising an annealing system to heat the thin film to reset it after exposure to the H₂S.

Statement 20. The system of any one of Statements 15-19, wherein the H₂S gas diffusion barrier is beryllium oxide.

The preceding description provides various examples of the systems and methods of use disclosed herein which may contain different method steps and alternative combinations of components. It should be understood that, although individual examples may be discussed herein, the present disclosure covers all combinations of the disclosed examples, including, the different component combinations, method step combinations, and properties of the system. It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the elements that it introduces.

For the sake of brevity, only certain ranges are explicitly disclosed herein. However, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present examples are well adapted to attain the ends and advantages mentioned as well as those that are inherent therein. The particular examples disclosed above are illustrative only and may be modified and practiced in different but equivalent manners apparent to those skilled in the art having the benefit of the teachings herein. Although individual examples are discussed, the disclosure covers all combinations of all of the examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of those examples. If there is any conflict in the usages of a word or term in this specification and one or more patent(s) or other documents that may be incorporated herein by reference, the definitions that are consistent with this specification should be adopted.

What is claimed is:

1. A method comprising:
conveying a fluid sampling tool into a wellbore wherein the fluid sampling tool comprises:
at least one probe to fluidly connect the fluid sampling tool to a formation in the wellbore; and
at least one passageway that passes through the at least one probe and into the fluid sampling tool;
drawing a formation fluid from a first sampling zone, as a fluid sample, through the at least one probe and through the at least one passageway;
passing the fluid sample over a hydrogen sulfide (H₂S) gas diffusion barrier that controls diffusion of a H₂S from the fluid sample to a thin film deposited on a measurement region, wherein the measurement region is part of the at least one passageway; and
analyzing the fluid sample in the fluid sampling tool for a target analyte.

2. The method of claim 1, wherein the thin film comprises at least one metal selected from a group of metals consisting of tin oxide alloyed doped with silver, chromium oxide alloyed doped with silver, indium tin oxide alloyed doped with silver, and any combination thereof.

3. The method of claim 1, wherein the thin film has a change of at least one property when the H₂S comes into contact with the thin film.

4. The method of claim 3, wherein the change of the at least one property of the thin film is proportional to a concentration of the H₂S.

5. The method of claim 1, wherein the H₂S gas diffusion barrier is beryllium oxide.

6. The method of claim 1, wherein the thin film changes optical properties when the H₂S comes into contact with the thin film.

7. The method of claim 1, further comprising identifying a change in optical properties of the thin film with an optical measurement tool, wherein the change in optical properties is proportional to a concentration of the H₂S, and wherein the concentration of the H₂S is determined from a linear, multivariate, or non-linear calibration model.

8. The method of claim 1, wherein one or more electrical properties of the thin film changes when the H₂S contacts the thin film.

9. The method of claim 8, wherein the one or more electrical properties comprise conductance, resistance, or inductance.

10. The method of claim 1, further comprising passing the fluid sample over a plurality of thin films.

11. The method of claim 10, wherein the plurality of thin films is disposed in an array or a pattern.

12. The method of claim 1, wherein the H₂S gas diffusion barrier is a homogeneous layer over the thin film.

13. The method of claim 1, wherein the H₂S gas diffusion barrier covers at least partially the thin film with layers of different thicknesses.

14. The method of claim 1, further resetting the thin film after exposure to the H₂S by annealing the thin film under exposure to a gas transported downhole in a sample chamber.

15. A system comprising a fluid sampling tool comprising:
at least one probe to fluidly connect the fluid sampling tool to a formation in a wellbore;
at least one passageway that passes through the at least one probe and into the fluid sampling tool;
a sensor section comprising a measurement region in fluid communication with the at least one passageway; and
a thin film deposited on the measurement region, wherein the thin film changes at least one of its physical properties upon exposure to a hydrogen sulfide (H₂S) found in the at least one passageway and wherein the thin film is covered at least partially by a H₂S gas diffusion barrier.

16. The system of claim 15, wherein the thin film comprises at least one metal selected from a group of metals consisting of tin oxide alloyed doped with silver, chromium oxide alloyed doped with silver, indium tin oxide alloyed doped with silver, and any combination thereof.

17. The system of claim 15, wherein the change of the at least one of the physical properties of the thin film is proportional to a concentration of the H₂S.

18. The system of claim 15, further comprising at least one sampling chamber filled with a gas used to reset the thin film after exposure to the H₂S.

19. The system of claim 15, further comprising an annealing system to heat the thin film to reset it after exposure to the H₂S.

20. The system of claim 15, wherein the H₂S gas diffusion barrier is beryllium oxide.
